(19) United States
(12) Patent Application Publication
(10) Pub. No.: US 2025/0002864 A1
(43) Pub. Date: Jan. 2, 2025

(54) RECOMBINANT ANIMAL CELL HAVING ATF7IP AND/OR SETDB1 GENE KNOCKOUT FOR PRODUCTION OF PRODUCT OF INTEREST AND USE THEREOF

(71) Applicant: KOREA ADVANCED INSTITUTE OF SCIENCE AND TECHNOLOGY, Daejeon (KR)

(72) Inventors: Su Hyun KIM, Daejeon (KR); Gyun Min Lee, Daejeon (KR)

(73) Assignee: KOREA ADVANCED INSTITUTE OF SCIENCE AND TECHNOLOGY, Daejeon (KR)

(21) Appl. No.: 18/754,349

(22) Filed: Jun. 26, 2024

(30) Foreign Application Priority Data
Jun. 28, 2023 (KR) 10-2023-0083606
Oct. 19, 2023 (KR) 10-2023-0140612

(51) Int. Cl. C12N 5/071

(52) U.S. Cl. CPC C12N 5/0682; C12N 2510/02

(57) ABSTRACT

Disclosed are a recombinant animal cell line having an ATF7IP gene and/or a SETDB1 gene knockout for production of a product of interest and use thereof, in particular, a recombinant animal cell line having knockout of at least one of an ATF7IP gene and/or a SETDB1 gene and a method of producing a product of interest including a therapeutic protein such as an antibody, a gene, or a vector, by using the same.

### CROSS-REFERENCE TO RELATED APPLICATION

This application is based on and claims priority under 35 U.S.C. § 119 to Korean Patent Application Nos. 10-2023-0083606, filed on Jun. 28, 2023, and 10-2023-0140612, filed on Oct. 19, 2023, in the Korean Intellectual Property Office, the disclosures of which are incorporated by reference herein in their entirety.

### BACKGROUND

### 1. Field

The disclosure relates to a recombinant animal cell in which an ATF7IP gene and/or a SETDB1 gene are knocked out for production of a product of interest, and use thereof, and in particular, to a recombinant animal cell having knockout of at least one of an ATF7IP gene and a SETDB1 gene and a method of producing the product of interest such as a therapeutic protein, e.g., an antibody, a gene, or a vector.

This study was conducted with the support of the Samsung Future Technology Promotion Project (Project No.: SRFC-MA1901-09).

### 2. Description of the Related Art

Mammalian cells including Chinese hamster ovary (CHO) cells and human embryonic kidney 293 (HEK293) cells have drawn attention as a large-scale production platform for therapeutic proteins. Extensive approaches have been attempted to improve product yield and to successfully achieve g/L scale titer in the production of biopharmaceuticals such as monoclonal antibodies. However, the emergence of structurally complex recombinant proteins such as bispecific antibodies (bsAbs) and engineered protein scaffolds has made the biopharmaceutical field increasingly heterogeneous. This trend has highlighted the necessity of genetically modified host cells that ensure optimal cost-effective production of therapeutic proteins such as antibodies.

Due to the challenge of achieving high-yield production of proteins with complex structures, the proteins are considered difficult-to-express (DTE) products, and thus there is a growing need for host cells for efficient production thereof. The expression of DTE proteins in CHO cells has been partially improved by using approaches such as engineering of secretary pathways, lipid metabolism, and transcription factors. However, productivity of DTE proteins, such as bispecific antibodies, is still as low as 1 g/L (Drug Discov. Today 20(7). 838-847 (2015)), and secretion in CHO cells is considered to act as a bottleneck (Bacteriology Advances 35(2017) 64-76). Therefore, there is a need to develop host cells by identifying a target that can efficiently enhance expression of DTE proteins and productivity thereof.

Recently, CRISPR/Cas9 library screening has been used to identify essential genes and targets for treatment of disease in various types of human cells mainly by using integration of a virus-mediated gRNA cassette. The recombinase-mediated cassette exchange (RMCE) system is an attractive option for delivering a gRNA, a single copy integration of which is required for unbiased and consistent expression thereof. The development of recombinant cell lines by using RMCE enables single-copy integration of transgenes at predefined sites by sorting non-fluorescent cells having a donor gene cassette, while avoiding random integration of gene cassettes with a promoter or poly(A) trap placed outside a landing pad (Hamaker and Lee, 2018, Curr. Opin. Chem. Eng. 22, 152-160). This strategy of cell line development has been highlighted in the production of therapeutic proteins because of homogenous and stable expression of transgenes (Srirangan et al., 2020, Crit. Rev. Biotechnol. 40, 833-851).

Accordingly, the inventors have developed an RMCE-based CRISPR/Cas9 screening system and have identified a novel engineering target for enhancing productivity of next-generation biopharmaceuticals, such as bispecific antibodies, currently known to be difficult to produce, in a genome-wide high-throughput screening, thereby completing the present disclosure.

### SUMMARY

Provided is a target gene related to productivity of a product of interest in a recombinant animal cell.

Provided is a recombinant animal cell having increased productivity of a product of interest as compared to a parent cell.

Provided is a method of producing a product of interest by using a recombinant animal cell with increased productivity of the product of interest as compared to a parent cell.

Additional aspects will be set forth in part in the description which follows and, in part, will be apparent from the description, or may be learned by practice of the presented embodiments of the disclosure.

According to an aspect of the disclosure, a recombinant animal cell having knockout of at least one of ATF7IP gene and Setdb1 gene is provided.

In an embodiment of the present disclosure, the recombinant animal cell may have knockout of ATF7IP gene, Setdb1 gene, or both the ATF7IP gene and the Setdb1 gene.

As used herein, the term “recombinant animal cell” refers to a cell which is genetically modified compared to a native or parent animal cell by transfection, genome editing or other genetic manipulation. Because recombinant animal cells provide human-like post-translational modification, the cells have been developed and used as a useful expression platform for production of products of interest such as therapeutic proteins. As a platform for production of products of interest, attempts have been made to improve cell viability, growth rate, productivity of the products of interest, and other relevant features of recombinant animal cells. An animal cell refers to a cell derived from an animal that can be used for expression of a product of interest and includes a mammalian cell, for example, a CHO cell, and a HEK293 cell.

ATF7IP gene and SETDB1 gene were screened as those associated with cell-specific antibody productivity by genome-wide screening in CHO cells for production of therapeutic proteins such as a bispecific antibody, and knockout of at least one of them in CHO cells were found to enhance cell-specific productivity, i.e., specific productivity of the bispecific antibody.

ATF7IP and SETDB1 respectively produced by expression of ATF7IP gene (RefSeq accession number: NC_048601.1) and SETDB1 gene (RefSeq accession number: NW_023276806.1) are known as histone modifiers and interact each other as binding partners to form an ATF7IP-SETDB1 complex, thereby depositing H3K9me3(histone H3 ‘Lys-9’ trimethylation) associated with epigenetic repression of transgenes (Fujita et al., Mol. Cell. Biol. 23, 2834-43 (2003); Wang et al., Mol. Cell 12, 475-487 (2003)). The inventors have found that knockout of at least one of the ATF7IP and SETDB1 genes led to decrease in epigenetic repression by H3K9me3 and increase in productivity of an antibody encoded by a transgene in recombinant CHO cells, and thus selected these genes as targets for enhancing productivity of recombinant animal cells.

The ATF7IP gene and SETDB1 gene are known to be associated with epigenetic repression of transgenes (Cancer Immunol Res 2021;9:1298-315). However, due to a post-transcriptional or post-translational bottleneck in CHO cells, an increase in a transcript level of a transgene or an intracellular protein level thereof shows a non-linear relationship with cell-specific productivity of proteins secreted from the cell (Biotechnology Advances 35 (2017) 64-76), and thus the relationship between the ATF7IP and SETDB1 genes and the productivity of DTE (difficult-to-express) products such as bispecific antibodies has not been known.

Herein, “ATF7IP” gene and “SETDB1” gene are used interchangeably with “Atf7ip” gene and “Setdb1” gene, respectively.

As used herein, the term “knockout” means that a subject gene is physically removed from a genome such that the gene does not function, and includes removal of the gene by homologous recombination by using CRISPR/Cas9, but without limitation thereto, knockout of the gene may be achieved by using any other method known in the art.

In an embodiment of the present disclosure, the recombinant animal cell may be a cell having increased productivity of a product of interest as compared to a parent cell from which the recombinant animal cell is derived with at least one of the ATF7IP gene and the Setdb1 gene knocked out.

As used herein, the term “a parent cell” refers to a cell before desired modification, such as a gene knockout, occurs. For example, a parent cell of a recombinant animal cell having knockout of at least one of Atf7ip gene and Setdb1 gene, is identical to the recombinant animal cell except for the knockout of at least one of the Atf7ip gene and the Setdb1 gene.

In an embodiment of the present disclosure, the recombinant animal cell may be a cell having higher cell-specific productivity than that of a parent cell from which at least one of the Atf7ip gene and the Setdb1 gene were to be knocked out.

In an embodiment of the present disclosure, the recombinant animal cell may include a gene encoding a product of interest.

As used herein, the term “a product of interest” refers to a substance to be produced by culturing a recombinant cell that includes a gene encoding the same, genes related to a metabolic pathway including biosynthesis thereof, or modification thereof, and includes proteins, nucleic acids, viruses, and the like, but is not limited thereto.

In an embodiment of the present disclosure, the product of interest may be a therapeutic protein, a gene therapy product, a cell therapy product, a vector, or a vaccine, but is not limited thereto.

In an embodiment of the present disclosure, the product of interest may be an antibody.

In an embodiment of the present disclosure, the antibody may be a bispecific antibody.

In an embodiment of the present disclosure, the gene encoding a product of interest may be inserted into a genome or present as a vector in the recombinant animal cell.

In an embodiment of the present disclosure, the recombinant animal cell may be produced by targeted integration into a landing pad of an expression cassette of a gene encoding a product of interest.

As used herein, the term “landing pad” refers to a site-specific recognition sequence or site-specific recombination site, e.g., attP site, safely integrated into a genome in a host cell and is used for site-specific or targeted integration of an exogenous sequence such as a gene encoding a product of interest.

In an embodiment of the present disclosure, the product of interest may be a product produced in a cell and secreted to the outside.

In an embodiment of the present disclosure, the recombinant animal cell may be a CHO cell.

In an embodiment of the present disclosure, the recombinant animal cell may be a CHO-K1 cell.

In an embodiment of the present disclosure, the recombinant animal cell may be a CHO-S cell.

According to another aspect of the disclosure,
a method for producing a product of interest, comprising
culturing a recombinant animal cell having a gene encoding a product of interest and knockout of at least one of an Atf7ip gene and a Setdb1 gene, and
recovering the product of interest from a culture is provided.

In the method, the product of interest may be a therapeutic protein, a gene therapy product, a cell therapy product, a vector, or a vaccine.

In an embodiment of the present disclosure, the recombinant animal cell may be cultured for a longer time than a parent cell in which the Atf7ip gene and the Setdb1 gene are not knocked out and produce a larger amount of the product of interest than the parent cell.

In an embodiment of the present disclosure, the recombinant animal cell may be a CHO cell, and the product of interest may be an antibody.

In an embodiment of the present disclosure, the recombinant animal cell may be a CHO cell, and the product of interest may be produced in the cell and secreted to the outside.

In an embodiment of the present disclosure, the product of interest may be a therapeutic protein, a gene therapy product, a cell therapy product, a vector, or a vaccine, but is not limited thereto.

In an embodiment of the present disclosure, the culturing of the recombinant animal cell may be performed by adherent culture or suspension culture, and may be performed by batch culture, fed-batch culture, or continuous culture.

The culturing of the recombinant animal cell may be performed in an appropriate condition including culture medium, temperature, and humidity, for production of the product of interest, which may be appropriately selected by any one of ordinary skill in the art to which the present disclosure pertains.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A to 1C show a schematic diagram of establishment of a recombinase-mediated cassette exchange (RMCE)-based genome-wide CRISPR knockout screening platform, and representation of gRNA library distribution.

FIG. 1A illustrates the generation of a CHO-bsAb master cell line (MCL) harboring mCherry and hygromycin-resistance genes flanked by attP and attP^{mut} recombination sites by CRISPR/Cas9-based targeted integration. The gRNA library plasmid contains the attB and attB^{mut} sites, which allow subsequent RMCE in the presence of Bxb1 recombinase. gRNA library cell pool was generated by delivering the gRNA library to CHO-bsAb cells via RMCE, and subsequently, knockout library cell pool was generated via transient Cas9 transfection. FIG. 1B shows cumulative percentages of gRNA reads, and FIG. 1C shows read count distribution of gRNAs and genes. Plasmid library is shown in open circle, cell-based library is shown in closed circle, and knockout library is shown in dotted circle.

FIGS. 2A to 2E show identification of novel targets through FACS-based CRISPR screening. FIG. 2A is a schematic diagram of cold-capture assay-based FACS screening, and FIG. 2B shows isolation of high-productivity cells via two rounds of FACS. The first round of FACS aimed the top 1% PE⁺ population, and the second round of FACS aimed the top 13.6±1.7% PE⁺ population of first-round sorted pool, which corresponds to the top 0.5% population of a knockout library cell pool. FIG. 2C shows a histogram of FACS and relative MFI of a surface-stained knockout library cell pool (KO library pool) and a sorted cell pool (After FACS). FIG. 2D shows a bubble plot illustrating the hits from the screening. Bubble size is proportional to the number of significantly enriched gRNAs. High-scoring 21 genes are shown in open and hatched circle, and genes related to histone methylation are shown in hatched circle together with the names of the genes. FIG. 2E shows validation of the screening hit using an all-in-one CRISPR/Cas9 plasmid: relative MFI of surface-stained knockout cell pools targeting 13 hits. Genes related to histone methylation are shown in hatched bar. Asterisks (*) indicate the significant difference compared to the non-target (NT) control. Error bars in the plot represent the standard deviations of three independent experiments. An unpaired two-tailed t-test was used to determine the significance of mean difference. *P<0.05; **P<0.01; ****P<0.0001.

FIGS. 3A to 3D show that impairment of ATF7IP and SETDB1 enhanced productivity in CHO-bsAb cells according to an embodiment of the present disclosure. FIG. 3A is a schematic diagram illustrating the function of ATF7IP and SETDB1. FIG. 3B is a FACS histogram overlay representing a surface-stained NT control and knockout cell pools. FIG. 3C shows western blot analysis of ATF7IP, SETDB1, β-actin, and H3K9me3 in the NT control and knockout cell pools. FIG. 3D shows (d) viable cell concentration (VCC), (e) antibody titer, (f) cell-specific productivity (q_{bsAb}) of the NT control and the knockout cell pools, (g) VCC, (h) antibody titer, and (i) q_{bsAb} of the NT control and the knockout clones. The dashed line in the box indicates the mean value, and the solid line in the box indicates the median value. The box limits indicate the 25^{th} and 75^{th} percentiles, and the whiskers indicate the 10^{th} and 90^{th} percentiles. All data points are plotted as open circles. n=6, 19, 13, and 15. VCC and antibody titer were measured on day 3. Asterisks (*) indicate significant differences compared to a non-target (NT) control. Error bars in the plots represent standard deviation of three independent experiments. An unpaired two-tailed t-test was used to determine the significance of the mean difference. n.s. P>0.05; *P<0.05; **P<0.01; ***P<0.001; ****P<0.0001.

FIGS. 4A to 4G show that ATF7IP and SETDB1 knockout CHO-bsAb clone according to an embodiment of the present disclosure exhibited enhanced productivity and transgene mRNA expression level. FIGS. 4A to 4C show profiles of cell growth, viability, and bsAb concentration of the NT control and the knockout clones, respectively, FIG. 4D shows q_{bsAb} calculated from day 2 to day 4, and FIG. 4E shows western blot analysis of ATF7IP, SETDB1, β-actin, and H3K9me3 in the NT control and the knockout clones. β-actin was used as a loading control. FIG. 4F shows relative mRNA expression levels of heavy chain and light chain measured on day 4, and the values were normalized to the NT control. FIG. 4G shows SEC analysis of ABL101. The expanded data for the high-molecular-weight portion is shown on the right. Asterisks (*) indicate significant differences compared to the non-target (NT) control. Error bars in the plot represent standard deviation of three independent experiments. An unpaired two-tailed t-test was used to determine the significance of the mean difference. *P<0.05; **P<0.01; ***P<0.001; ****P<0.0001.

FIGS. 5A to 5D show that impairment of ATF7IP and SETDB1 further enhanced productivity in CHO-mAb cells according to an embodiment of the present disclosure. FIGS. 5A and 5B show a FACS histogram overlay and relative MFI of surface-stained NT control and knockout cell pools, and FIG. 5C shows western blot analysis of ATF7IP, SETDB1, β-actin, and H3K9me3 in the NT control and knockout cell pools. β-actin was used as a loading control. FIG. 5D shows (d) VCC, (e) antibody titer, (f) cell-specific mAb productivity (q_{mAb}) of the NT control and the knockout cell pools, (g) VCC, (h) antibody titer, and (i) cell-specific mAb productivity (q_{mAb}) of the NT control and the knockout clones. The dashed line in the box indicates the mean value, and the solid line in the box indicates the median value. The box limits indicate the 25^{th} and 75^{th} percentiles, and the whiskers indicate the 10^{th} and 90^{th} percentiles. All data is shown as open circles. n=6, 19, 13, and 15. VCC and antibody titer were measured on day 3. Asterisks (*) indicate significant differences compared to a non-target (NT) control. Error bars in the plots represent standard deviation of three independent experiments. An unpaired two-tailed t-test was used to determine the significance of the mean difference. n.s. P>0.05; *P<0.05; **P<0.01; ***P<0.001; ****P<0.0001.

FIGS. 6A to 6H show that ATF7IP and SETDB1 knockout CHO-mAb clones according to an embodiment of the present disclosure exhibited enhanced productivity and transgene mRNA expression level without affecting the quality of products. FIGS. 6A to 6C show profiles of cell growth, viability, and mAb concentration of the NT control and the knockout clones, FIG. 6D shows q_{bsAb} calculated from day 2 to day 5, and FIG. 6E shows western blot analysis of ATF7IP, SETDB1, β-actin, and H3K9me3 in the NT control and the knockout clones. β-actin was used as a loading control. FIG. 6F shows relative mRNA expression levels of heavy chain and light chain measured on day 4. Values were normalized to the NT control. FIG. 6G shows SEC analysis of Rituximab, and FIG. 6H shows the relative abundance of N-glycosylation of Rituximab. Asterisks (*) indicate significant differences compared to a non-target (NT) control. Error bars in the plot represent standard deviation of three independent experiments. An unpaired two-tailed t-test was used to determine the significance of mean difference. G0: agalactosylated glycan without fucose; G0F: agalactosylated glycan with fucose; G1F(1,6) and G1F(1,3): monogalactosylated glycan with fucose; G2F: digalactosylated glycan with fucose; *P<0.05, **P<0.01, ***P<0.001, and ****P<0.0001.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the present disclosure will be described in more detail with reference to the following examples. However, the following examples are merely presented to exemplify the present disclosure and the scope of the present disclosure is not limited thereto.

### EXAMPLE 1: ESTABLISHMENT OF RMCE-BASED GENOME-WIDE CRISPR/CAS9 KNOCKOUT SCREENING PLATFORM IN BISPECIFIC ANTIBODY (BSAB)-PRODUCING CHO CELL LINE

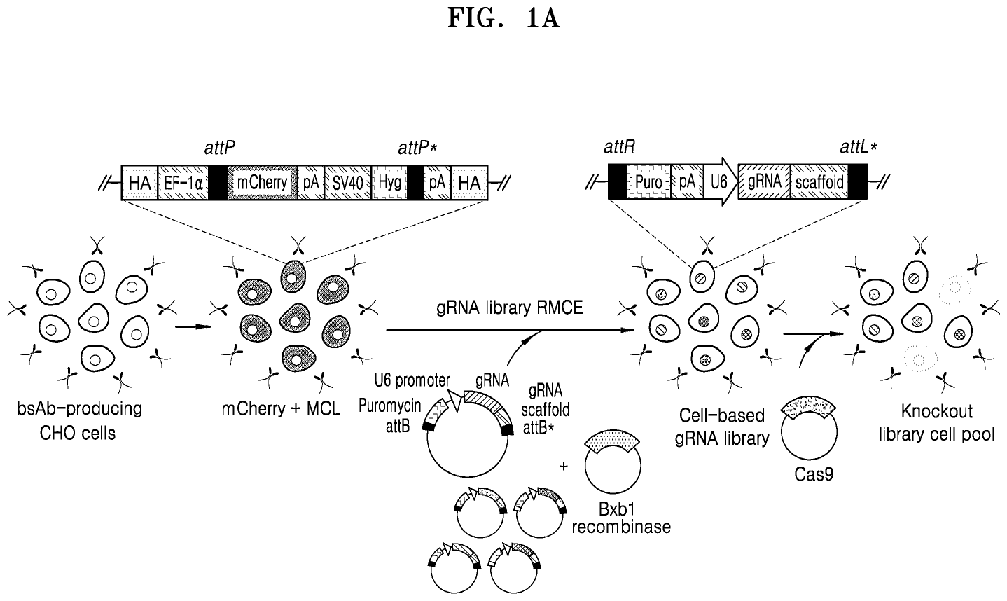

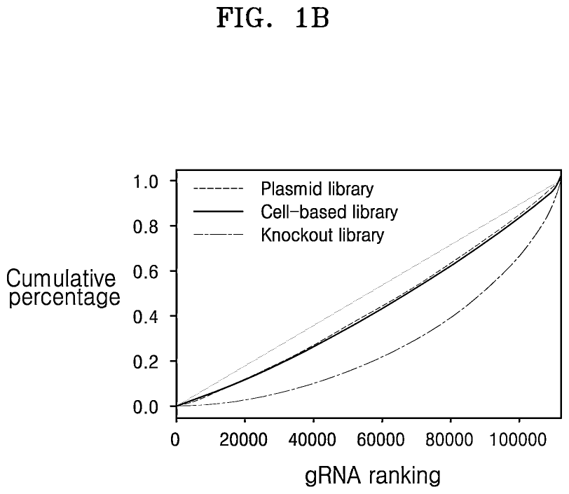

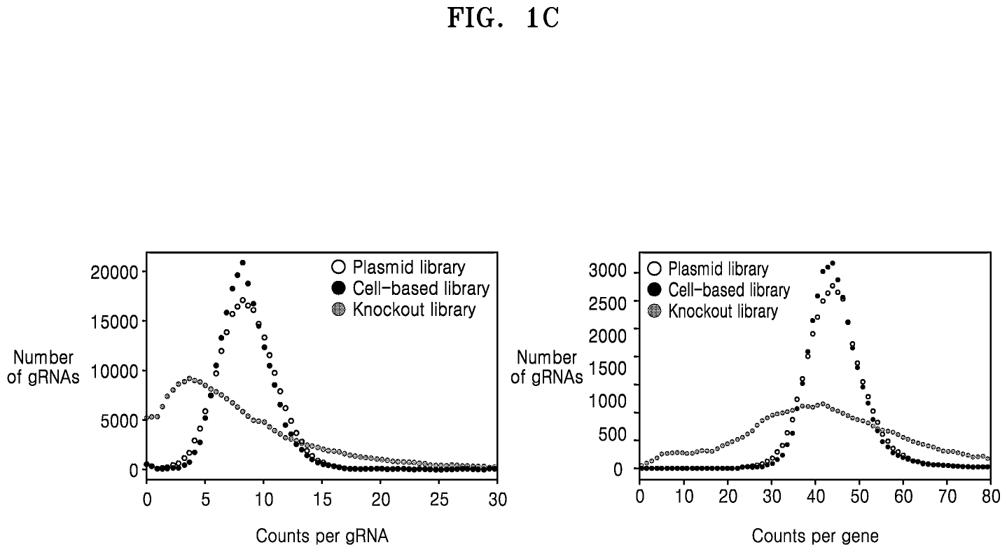

In order to screen targets related to productivity of a bispecific antibody, a recombinase-mediated cassette exchange (RMCE)-based CRISPR/Cas9 knockout screening platform was constructed in a CHO cell line producing ABL101 bsAb (CHO-bsAb). FIG. 1 shows a schematic diagram of RMCE-based CRISPR/Cas9 library screening.

### 1-1. Generation of Genome-Wide CRISPR gRNA Library

A gRNA library targeting the whole genome of a CHO-K1 cell was designed. Specifically, coding DNA sequence (CDS) in the whole genome of the CHO-K1 cell were analyzed to identify potential functional domains, and a total of 111,651 gRNAs targeting 21,585 genes were designed. Based thereon, a gRNA library plasmid pool was prepared and the distribution of the gRNA library was verified to construct a genome-wide gRNA library.

Specifically, the gRNA sequence was amplified by using a Phusion High-Fidelity PCR Master Mix (Thermo Fisher Scientific) and purified by a 2% agarose gel by using a NucleoSpin Gel and PCR Clean-up (Macherey-Nagel). According to the manufacturer's instructions, the gRNA sequence was cloned into an attB-Esp3I-BSD plasmid treated with FastDigest Esp3I enzyme (Thermo Fisher Scientific) (Xiong, K. et al., Cell Rep. Methods 1, 100062 (2021)) by using a Gibson Assembly Master Mix (New England Biolabs) to construct an attB-gRNA plasmid (attB-Puro-BGHpA-U6-gRNA (library)-gRNA_scaffold-attB^{mut}). The constructed gRNA library plasmid was transferred to Endura ElectroCompetent cells (Lucigen, Middleton, WI) by using a MicroPulser Electroporator (Bio-Rad, Hercules, CA) at a yield of 500 copies per gRNA for each library, and the obtained cells were incubated in an LB-ampicillin medium at 37° C. overnight. Then, the plasmid DNA was purified by using a NucleoBond Xtra Maxi EF Kit (Macherey-Nagel).

### Analysis of Distribution of gRNA Library

In order to identify whether the constructed gRNA library represents the whole genome of the CHO-K1 cell, the gRNA sequence was amplified in the library plasmid or genome DNA of the library cell, sequenced using NGS, and analyzed distribution of reads. By the NGS analysis, it was verified that the gRNA library cell pool showed an even distribution, representing the whole genome.

### 1.2. Generation of Knockout Cell Library

A knockout cell library was established by using the genome-wide gRNA library obtained in 1-1 above.

### Generation of MCL Expressing mCherry

A CHO-bsAb cell expressing mCherry was prepared by using an mCherry landing pad (HA-EF1α-attP-mCherry-pA-SV40-Hyg-attP^{mut}-pA-HA) containing homology arms for CRISPR/Cas9-mediated integration, attP/attP* sites flanking a mCherry reporter gene and a hygromycin resistance gene, and a promoter/poly(A)trap for efficient RMCE (Xiong, K. et al., Cell Rep. Methods 1, 100062 (2021)).

The CHO-bsAb cell line (ABL Bio, Korea) is a cell line obtained by engineering a CHO-S cell to express ABL101 bsAb targeting both BCMA and 4-1BB. In order to introduce the mCherry landing pad into the CHO-bsAb cell line (ABL Bio, Korea), the CHO-bsAb cell was transfected with the mCherry landing pad plasmid (U6-gRNA (mCherry)-gRNA_scaffold-CMV-Cas9-P2A-BSD-BGHpA), a gRNA plasmid targeting non-coding region (site T2) (U6-gRNA-gRNA_scaffold-CMV-Cas9-P2A-BSD-BGHpA), and a Cas9 plasmid (CMV-EGFP-T2A-Cas9-BGHpA-SV40-BSD-SV40pA) at a ratio of 1:1:1 (w/w) by using 293fectin (Thermo Fisher Scientific) according to the manufacturer's instructions. After two weeks of 800 μg/mL hygromycin (Clontech, San Jose, CA) selection, mCherry-expressing cells were single-cell sorted by using FACSAria Fusion (BD Biosciences, San Jose, CA). Clones were expanded and analyzed by the copy numbers of mCherry gene, mRNA expression, and mean fluorescence intensity (MFI) as described in ACS Synth. Biol. 7, 2148-2159, to select a clone exhibiting homogenous mCherry expression, which was used as “master cell line (MCL)”.

### Generation of Knockout Library Cell Pool by Using CRISPR/Cas9 Gene Editing

The master cell line (MCL) was transfected with the genome-wide CRISPR gRNA library including 111,651 gRNAs targeting 21,585 genes via RMCE by using an N-NLS-Bxb1 recombinase plasmid (CMV-Bxb1-NLS-BGHpA) (FIG. 1A). By puromycin selection, RMCE positive cells were enriched from 30.4% to 99.9% to generate a cell-based gRNA library, and two rounds of transient Cas9 transfection were conducted to prepare a knockout library cell pool.

Specifically, the number of cells required for transfection was calculated based on the measured value of RMCE efficiency to guarantee the coverage of 500 cells per gRNA. According to the manufacturer's instructions, a total of 3.0×10⁸ cells were transfected with gRNA library plasmid and Bxb1 recombinant plasmid at a ratio of 3:1 (w/w) by using 293fectin. Two days after the transfection, the cells were subcultured in the presence of 20 μg/mL puromycin (Sigma-Aldrich). After 18 days of puromycin selection, a cell-based gRNA library pool was generated and subjected to Cas9 transfection. According to the manufacturer's instructions, a total of 3.0×10⁸ cells were transfected with Cas9 plasmid (CMV-EGFP-T2A-Cas9-BGHpA-SV40-BSD-SV40pA) by using 293fectin. One day after transfection, the cells were subcultured in the presence of 20 μg/mL blasticidin (BSD, Sigma-Aldrich). After three days of selection, the cells were recovered in blasticidin-free fresh media. Four days after recovery, the cells were subjected to second round of Cas9 transfection, and selection by blasticidin was performed as described above. Ten days after the transfection, a knockout library cell pool was generated and a total of 5.6×10⁷ cells were harvested for genomic DNA extraction.

In order to verify the distribution of the gRNA library, gRNA sequences in the genomic DNA from the gRNA library cell pool and the knockout library cell pool were amplified and sequenced by next generation sequencing (NGS). The gRNA library cell pool exhibited an even distribution similar to the plasmid library with the coverage of 99.7% and the skew ratio of 1.98, indicating sufficient gRNA library distribution (FIG. 1B). Meanwhile, the gRNA library distribution of the knockout library cell pool was disrupted, implying that the gRNAs targeting genes essential for cell survival were depleted (FIG. 1C). Therefore, it was confirmed that the generated knockout library cell pool can be used for CRISPR screening.

### EXAMPLE 2. IDENTIFICATION OF TARGET GENES INCREASING PRODUCTIVITY PER UNIT CELL BY FACS-BASED SCREENING

In order to discover target genes related to an increase in cell-specific productivity of bispecific antibody (bsAb) in a CHO cell, highly productive cells were isolated from the genome-wide knockout library cell pool prepared in Example 1 and enrichment of the gRNAs in the cells was analyzed.

### 2.1. Isolation of CHO Cell with High Productivity and Selection of Target Gene Candidate

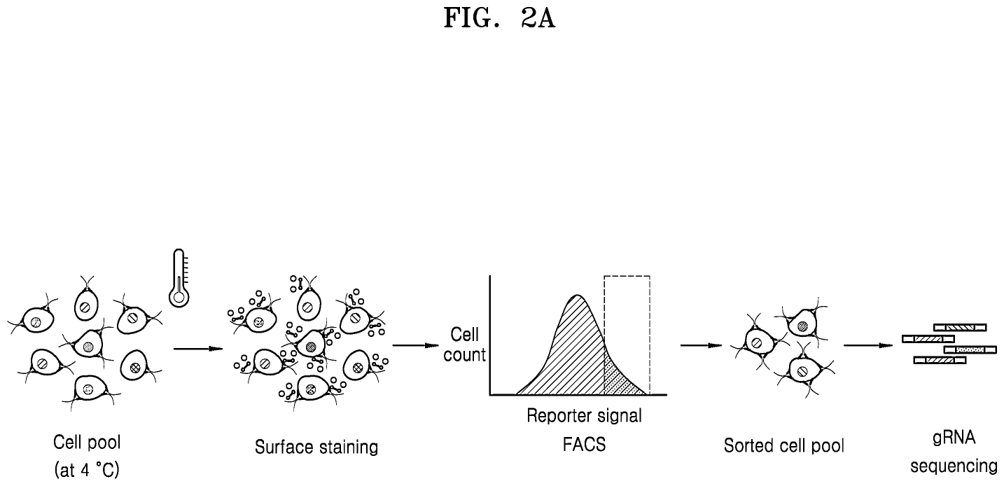

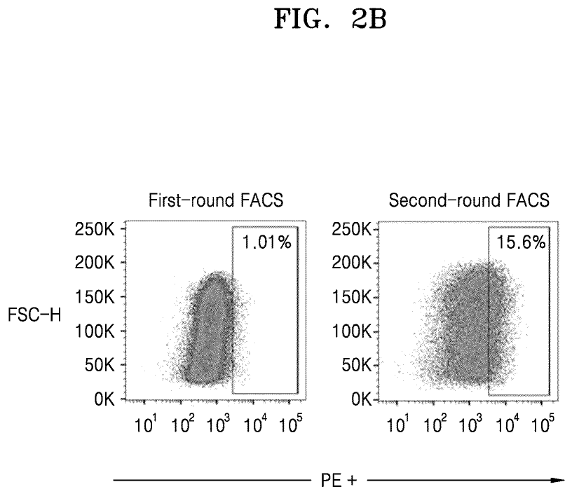

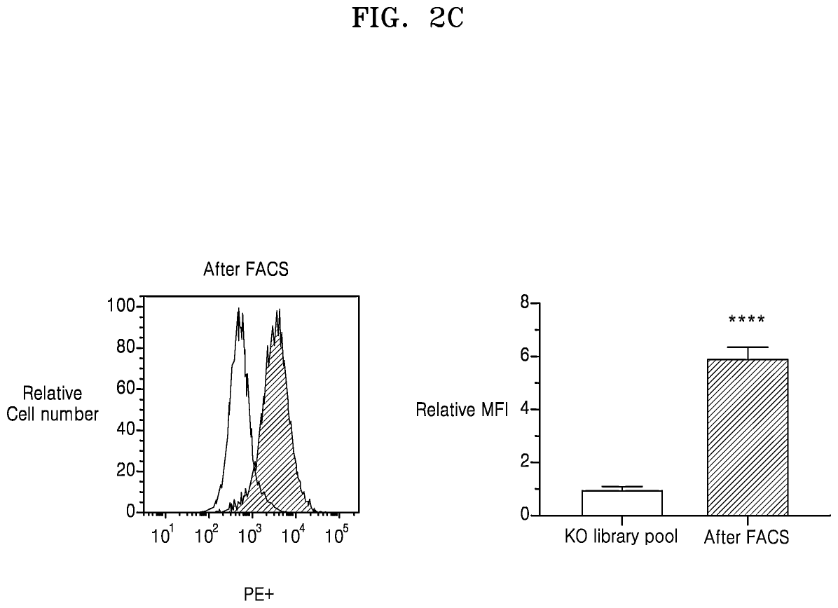

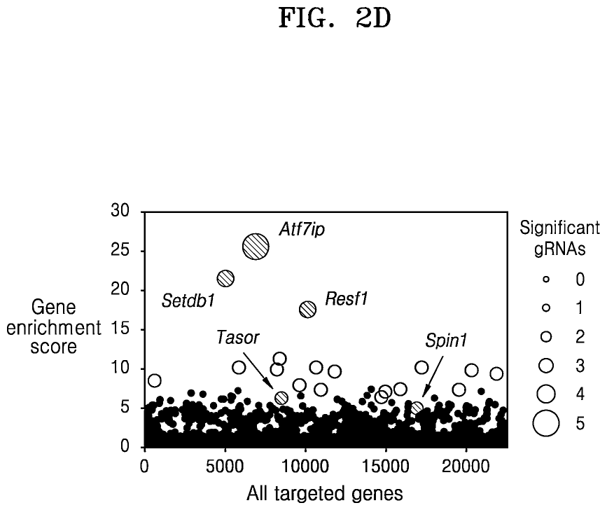

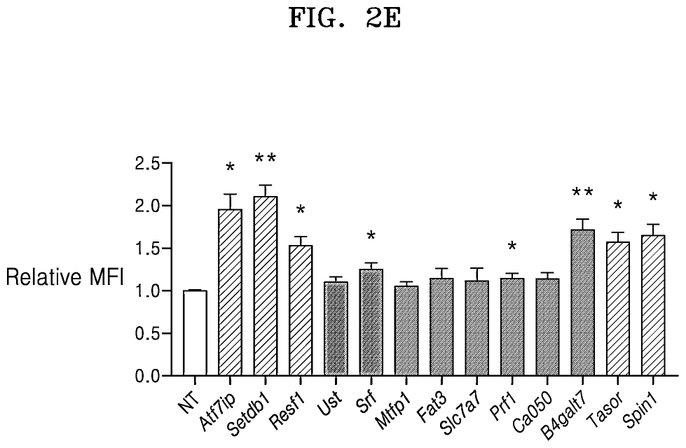

In order to uncover novel targets that drive high productivity of CHO cells on a genome-wide scale, cells showing high productivity of bsAb were isolated from the genome-wide knockout library cell pool prepared in Example 1 by using FACS-based cold-capture assay shown in FIG. 2A (Park et al., Appl. Microbiol. Biotechnol. 106, 3571-3582). In the first-round FACS, the top 1% population was sorted in three independent experiments (FIG. 2B). In the second-round FACS, the top 15.6%, 13%, and 12.3% populations of the first-round sorted triplicate pools were sorted respectively, and all of them corresponded to the top 0.5% population of the knockout library cell pool. Relative MFI was increased by 5.9±0.4 times after the two rounds of FACS (FIG. 2C). Genomic DNA was isolated from the sorted cell groups, and abundance of gRNA sequence was determined by NGS. In order to evaluate the distribution of gRNAs in the sorted cell pool against the knockout library cell pool, computational analysis was conducted as follows by using Platform-independent Analysis of PooLed screens using Python (PinAPL-Py).

### NGS Analysis

Genomic DNA was extracted from the cells by using an Exgene Blood SV kit (GeneAll Biotechnology, SEOUL, Korea) according to the manufacturer's instructions. In order to prepare NGS samples, PCR was performed with a total volume of 50 μl including 3 to 4 μg of genomic DNA per reaction by using a NEBNext Ultra II Q5 Master Mix (New England Biolabs, Ipswich, MA), (98° C. for 3 minutes; 22 cycles of 98° C. for 10 seconds, 60° C. for 30 seconds, and 72° C. for 30 seconds; and 72° C. for 5 minutes). PCR products were purified by using a NucleoSpin Gel and PCR purification kit (Macherey-Nagel) and indexed by using a TruSeq Nano DNA Library Prep kit (Illumina, San Diego, CA). The indexed library was quantified by qPCR by using an Illumina qPCR Quantification Protocol Guide (Illumina). The library size was determined by using a TapeStation D1000 ScreenTape (Agilent Technologies, Santa Clara, CA, USA) and sequenced on a HiSeq X Ten sequencer (Illumina).

### NGS Data Analysis

Computational data analysis was performed by using PinAPL-Py (Spahn et al., Sci. Rep. 7, 15854). All parameters were set to defaults and gene ontology was analyzed by using Metascape (Zhou et al., Nat. Commun. 10, 1523).

For high-scoring 21 genes, three or more gRNAs out of the 5 designed gRNAs were significantly enriched in the sorted pool and these gRNAs were considered as screen “hit” (FIG. 2D). Among them, 5 hits: Atf7ip, Setdb1, Kiaa1551(Resf1), Fam208a(Tasor), and Spin1, are associated with histone methylation, which mediates epigenetic repression. Interestingly, Atf7ip, for which all gRNAs were significantly enriched, and Setdb1 and Resf1, for which 4 gRNAs were significantly enriched, are all histone modifiers that specifically mediate histone H3 ‘Lys-9’ trimethylation (H3K9me3) associated with SETDB1.

To confirm results of the screening, knockout cell pools for the 5 hits and other high-scoring 8 hits were generated by using all-in-one CRISPR/Cas9 plasmids. The effectiveness of the all-in-one CRISPR/Cas9 system was verified by using mCherry⁺ MCL cell line and mCherry-targeting all-in-one CRISPR/Cas9 plasmid.

### 2.2. Generation of Knockout Cell Pool and Clones for Target Gene Candidates

In order to generate a knockout cell pool in the CHO-bsAb cell line, the cells were seeded at 1×10⁶ cells/mL in a 12-well plate containing 1 mL SFM4Transfx-293 (HyClone) supplemented with 4 mM glutamine and transfected with all-in-one CRISPR/Cas9 plasmids (U6-gRNA(target_gene)-gRNA_scaffold-CMV-Cas9-P2A-BSD-BGHpA) targeting each of the genes by using 293fectin, according to the manufacturer's instructions. After 48 hours, the transfected cells were treated 150 g/mL blasticidin for 3 days and recovered without blasticidin for 9 days.

In addition, for further validation, ATF7IP, SETDB1 and ATF7IP/SETDB1 double knockout cell pools were generated in CHO-bsAb and CHO-mAb cell lines. According to the manufacturer's instructions, cells were seeded at 1×10⁶ cells/mL in a 6-well plate containing 3 mL of SFM4Transfx-293 (HyClone) supplemented with 4 mM glutamine and transfected with all-in-one CRISPR/Cas9 plasmids (U6-gRNA(target_gene)-gRNA_scaffold-CMV-Cas9-P2A-BSD-BGHpA) by using 293fectin. After 48 hours, the transfected CHO-mAb cell lines were treated with 75 μg/mL blasticidin, and the transfected CHO-bsAb cell lines were treated with 150 μg/mL blasticidin.

For the double knockout cell pools, the cells were transfected with Atf7ip-targeting all-in-one CRISPR/Cas9 plasmid (U6-gRNA(Atf7ip)-gRNA_scaffold-CMV-Cas9-P2A-Puro-BGHpA) and Setdb1-targeting Puro all-in-one CRISPR/Cas9 plasmid (U6-gRNA(Setdb1)-gRNA_scaffold-CMV-Cas9-P2A-Puro-BGHpA) at a ratio of 1:1 (w/w), and further treated with 2 μg/mL puromycin during selection. After 3 days of selection, the cells were recovered for 9 days in the absence of puromycin. 5×10⁶ cells were collected for TIDE (Tracking of Indels by Decomposition) analysis (Brinkman et al., Nucleic Acids Res. 42, e168-e168). Clones were generated from the knockout cell pools by limiting dilution at a concentration of 0.5 cells/well and confirmed by TIDE analysis.

The effectiveness of the all-in-one CRISPR/Cas9 system was verified by using the mCherry⁺ MCL cell line and the mCherry-targeting all-in-one CRISPR/Cas9 plasmid (U6-gRNA(mCherry)-gRNA_scaffold-CMV-Cas9-P2A-BSD-BGHpA). Specifically, knockout efficiency was confirmed by selection at various concentrations of blasticidin (BSD). The mCherry⁺ MCLs were transfected with mCherry-targeting all-in-one CRISPR/Cas9 plasmids. After 48 hours, the transfected cells were treated with various concentrations of blasticidin for 3 days and recovered without blasticidin for 9 days, and then mCherry knockout efficiency was measured by flow cytometry. A total of 13 knockout cell pools were cultured in a 6-well plate for 3 days and subjected to cold-capture assay. Overall, 8 out of the 13 knockout cell pools showed a significant increase in fluorescence intensity compared to non-target (NT) control (FIG. 2E). Particularly, the fluorescence intensity increased by 1.5 times or more when the 5 genes related to histone methylation, i.e., Atf7ip, Setdb1, Kiaa1551(Resf1), Fam208a(Tasor), and Spin1, were knocked out. Taken together, the genome-wide FACS-based CRISPR screening shows that knockout of histone modifiers related to epigenetic repression leads to high productivity in CHO cells.

From this screening, 5 novel targets for high productivity of CHO cells, Atf7ip, Setdb1, Kiaa1551(Resf1), Fam208a(Tasor), and Spin1, were identified and selected.

### EXAMPLE 3. IMPROVEMENT OF Q_{BSAB} OF CHO-BSAB CELL BY ATF7IP AND/OR SETDB1 KNOCKOUT

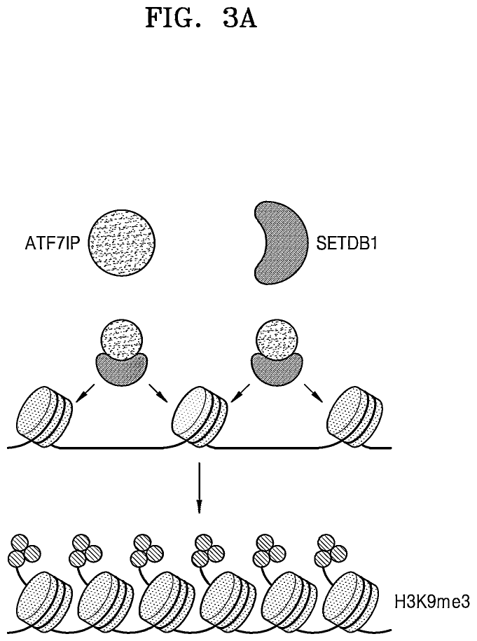

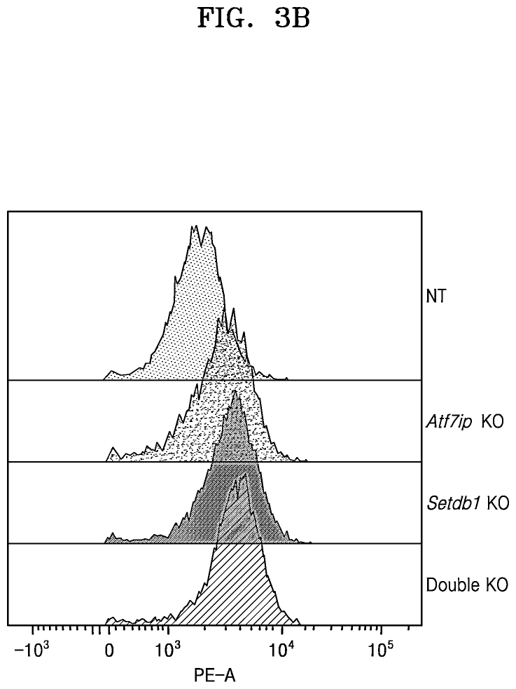

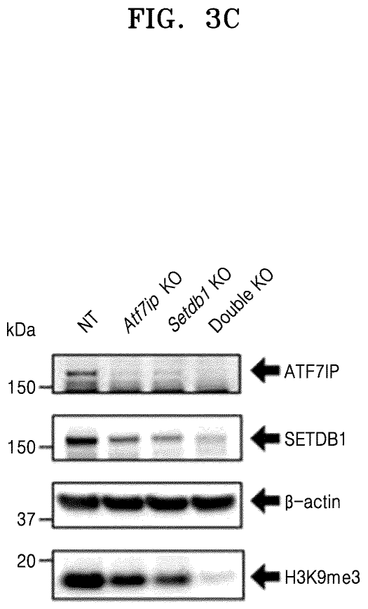

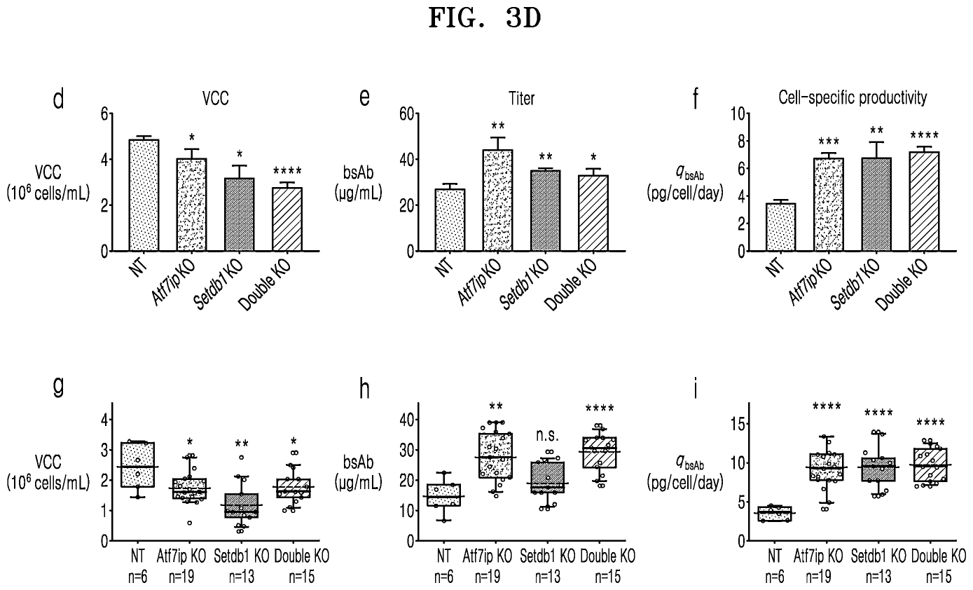

Atf7ip and Setdb1 were clearly identified as targets related to productivity of CHO cells both in the screening and the verification experiments performed in Example 2. ATF7IP and SETDB1 are known to interact each other as binding partners to deposit H3K9me3 and mediate transcriptional repression at heterochromatic loci (FIG. 3A) (Fujita et al., Mol. Cell. Biol. 23, 2834-43).

### 3.1. Preparation of Atf7ip and/or Setdb1 Knockout Cell Pools

In order to further investigate the effect of depletion of the ATF7IP-SETDB1 complex, as described in Example 2, the Atf7ip/Setdb1 double knockout cell pools were generated by using Atf7ip all-in-one CRISPR/Cas9 plasmid (U6-gRNA(Atf7ip)-gRNA_scaffold-CMV-Cas9-P2A-BSD-BGHpA) and Setdb1-Puro-all-in-one CRISPR/Cas9 plasmid (U6-gRNA(Setdb1)-gRNA_scaffold-CMV-Cas9-P2A-Puro-BGHpA).

All three knockout cell pools, i.e., Atf7ip knockout, Setdb1 knockout, and Atf7ip/Setdb1 double knockout, showed higher fluorescence intensity than that of the NT control (FIG. 3B). Knockout efficiency of each pool was assessed by TIDE analysis. In addition, protein expression of each target gene was confirmed by western blot analysis as described below (FIG. 3C). Notably, targeting ATF7IP resulted in a decrease in the level of SETDB1, and targeting SETDB1 resulted in a decrease in the level of ATF7IP, indicating that ATF7IP and SETDB1 are responsible for maintaining each other's stability (Timms et al., Cell Rep. 17, 653-659). All three knockout cell pools showed a decrease in H3K9me3 level compared to the NT control.

### 3.2. Cell Growth and Bispecific Antibody Titer

In order to evaluate the effect of gene knockout on cell growth and production of bispecific antibody (bsAb), the knockout cell pools were cultured in a 6-well plate and viable cell concentration (VCC) and bsAb titer were measured on day 3.

### Batch Culture

Specifically, three knockout cell pools were respectively seeded into a 125 ml Erlenmeyer flask containing 30 mL of a culture medium at a density of 0.5×10⁶ cells/mL and cultured at 110 rpm, 37° C., 85% humidity, and 5% CO₂. Cell concentration and viability were measured every day, and supernatants were collected every day and stored at −70° C. for further analysis.

### Western Blot Analysis and Measurement of bsAb Concentration

Samples for western blot analysis were prepared on day 3 from the batch culture. Western blot analysis was performed as previously described (Metab. Eng. 69, 73-86). Antibodies were purchased respectively and concentrations of the antibodies were measured by enzyme-linked immunosorbent assay (Biotechnol. Bioeng. 58, 73-84).

### Quantitative Real-Time Polymerase Chain Reaction (qRT-PCR)

Cells were sampled on day 4 from the batch cultures. According to the manufacturer's instructions, total RNA was extracted by using a Hybris-R RNA extraction kit (GeneAll Biotechnology). According to the manufacturer's instructions, cDNA was synthesized from the extracted RNA by using a High-Capacity cDNA Reverse Transcription Kit (Applied Biosystems, Foster City, CA). Genomic DNA was extracted as previously described. A qRT-PCR was performed to evaluate the relative mRNA levels and gene copy numbers (Sci. Rep. 8, 5361). Relative expression levels were calculated based on the ΔΔCT method and normalized to GAPDH.

Compared to the NT control, all three knockout cell pools showed a decrease in VCC (d in FIG. 3D) and an increase in bsAb titer (e in FIG. 3D), but the extent was different from each other. The Atf7ip knockout (6.8±0.3 pg/cell/day), Setdb1 knockout (6.9±1.1 pg/cell/day) and Atf7ip/Setdb1 double knockout (7.3±0.3 pg/cell/day) cell pools had q_{bsAb} 1.9-fold, 2.0-fold, and 2.1-fold higher than that of the NT control (3.5±0.2 pg/cell/day), respectively (f in FIG. 3D). As knockout of ATF7IP and SETDB1 showed a detrimental effect on cell growth, knockout cells may be diluted from the population upon prolonged culture. Thus, we generated isogenic clones of the Atf7ip knockout, the Setdb1 knockout, and the Atf7ip/Setdb1 double knockout by limiting dilution. Indels of each clone were assessed by TIDE analysis, and all clones with verified gene disruption were cultured in a 6-well plate, and then VCC and bsAb titers were measured on day 3. Atf7ip knockout and Atf7ip/Setdb1 double knockout clones showed a decrease in VCC (g in FIG. 3D) and an increase in titer (h in FIG. 3D), compared to the NT control clones. Setdb1 knockout clone exhibited a more significant suppression in cell growth and had similar or higher mean titer, although the difference in titer was not statistically significant. However, the mean qbsAb was significantly increased in all of the three types of knockout cells, which is consistent with the results observed in the knockout cell pool (I in FIG. 3D). The mean q_{bsAb} of Atf7ip knockout (9.4±2.8 pg/cell/day), Setdb1 knockout (9.4±2.7 pg/cell/day), and Atf7ip/Setdb1 double knockout (9.7±2.1 pg/cell/day) clones was 2.6-fold, 2.6-fold, and 2.7-fold higher than that of the NT control (3.5±0.8 pg/cell/day), respectively.

In conclusion, eliminating ATF7IP-SETDB1 complex improved the qosAb in the CHO-bsAb cells, verifying the effective screening of the target genes to improve productivity of the CHO cells.

### 3.3. Productivity and mRNA Expression Level

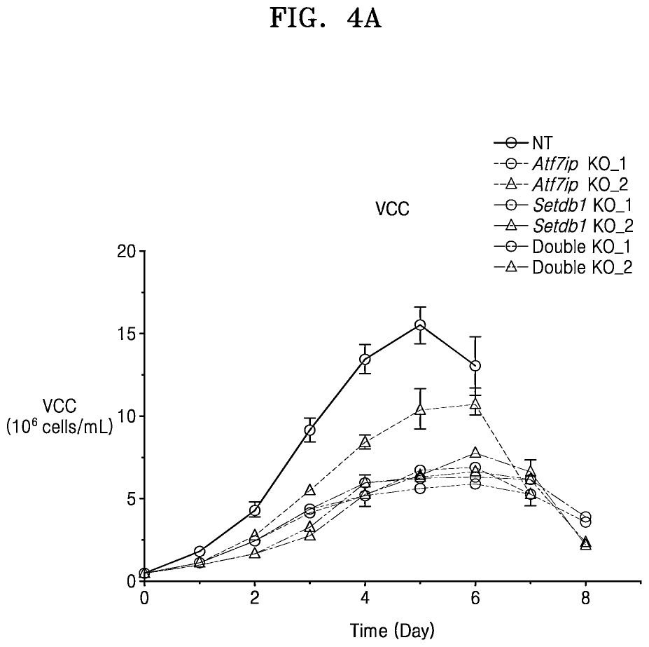

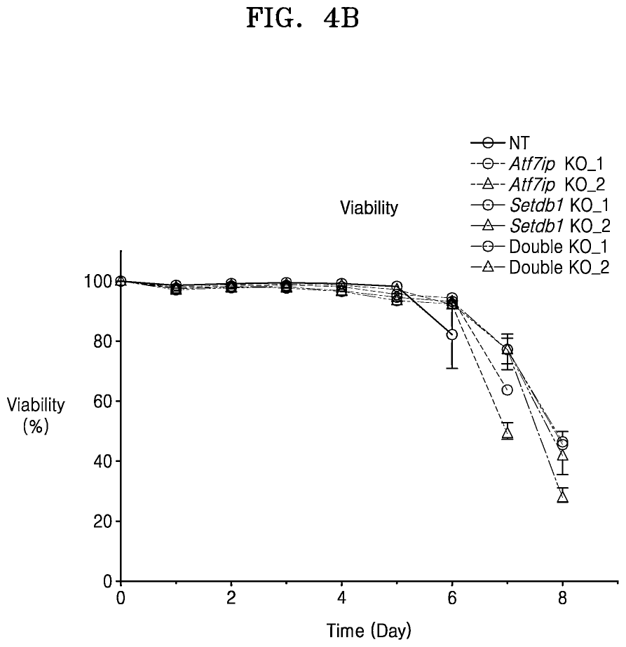

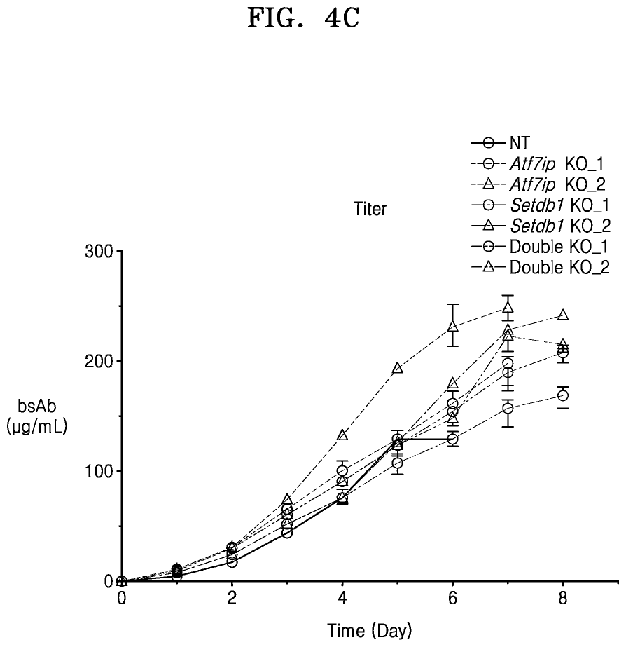

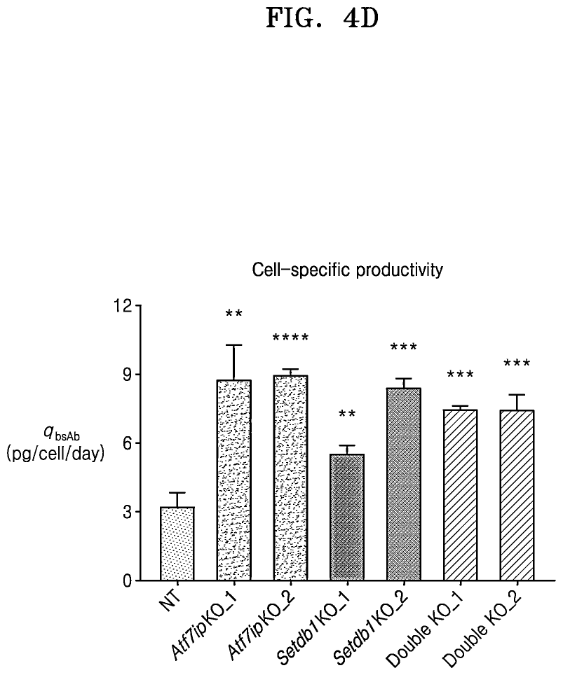

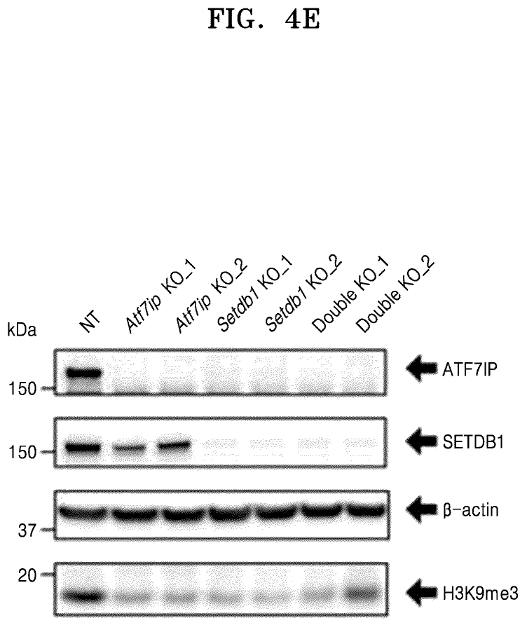

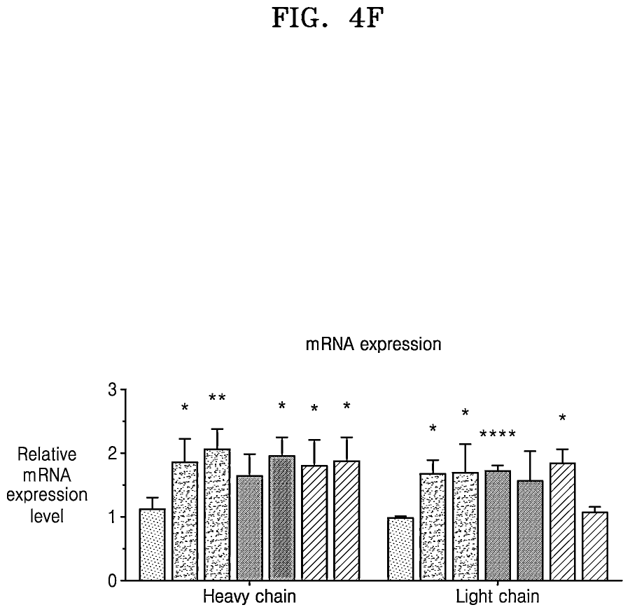

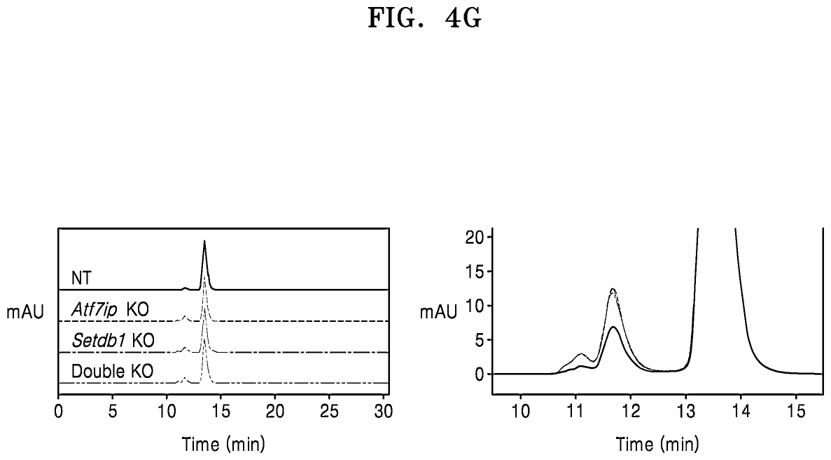

To further characterize the effect of depletion of ATF7IP and SETDB1 on production of bsAb in batch culture, the top two high-productivity clones from each knockout pool were cultivated in a 125 mL Erlenmeyer flask containing 30 mL of a culture medium. For comparison, the highest producing NT control clone was selected. Compared to the NT clone, knockout clones showed reduced cell growth, but maintained the cell viability longer (FIGS. 4A and 4B). As expected, knockout clones exhibited higher production of bsAb (FIG. 4C). The maximum bsAb concentrations of the Atf7ip knockout, Setdb1 knockout, and Atf7ip/Setdb1 double knockout clones were 1.5 to 1.9-fold, 1.3 to 1.9-fold, and 1.6 to 1.7-fold higher than that of the NT clone, respectively. The q_{bsAb} of the Atf7ip knockout, Setdb1 knockout, and Atf7ip/Setdb1 double knockout clones from day 2 to day 4 were 2.7-fold, 1.7 to 2.6-fold, and 2.3-fold higher than that of the NT clone, respectively (FIG. 4D).

Western blot analysis showed that each gene of each clone was successfully knocked out (FIG. 4E). Setdb1 knockout clone showed little expression of ATF7IP, and the SETDB1 level was reduced in the Atf7ip knockout clone showed a reduced level of SETDB1. In addition, the H3K9me3 level was reduced in all of the knockout clones. Therefore, we speculated that the increase in productivity may be attributed to the increase in transgene mRNA expression.

To measure relative mRNA expression levels of heavy chain and light chain, the cells were sampled from the cultures on day 4, and qRT-PCR was performed as described in Example 3. The knockout clones exhibited higher mRNA expression levels of the heavy chain and light chain than that of the NT clone (FIG. 4F).

### 3.4. Quality of Antibody Produced by Knockout Cell

To assess the quality of antibodies produced by the knockout cells, the antibodies were purified from the culture and size exclusion chromatography (SEC) was performed.

On day of the batch culture, culture supernatants were sampled. ABL101, a bispecific antibody produced by the knockout cells, was purified by protein A chromatography using a MabSelect™ SuRe™ (Cytiva, Marlborough, MA, USA). For the ABL101, SEC was performed using a TSKgel G3000SW-XL column (250 Å, 5 μm, 7.8×300 mm, Tosoh Bioscience, Tokyo, Japan), and connected to an HPLC system (1260 Infinity II Bio LC, Agilent Technologies). A mobile phase consisted of 40 mM sodium phosphate, 0.4 M sodium perchlorate, and pH 6.8 buffer. The signal was monitored at a wavelength of 280 nm.

To assess a quality attribute of bsAb, the proportion of high molecular weight (HMW) derived from scFv aggregates was determined by SEC (FIG. 4G). In comparison with the NT control clone, the HMW proportion of the knockout clone was slightly increased probably because of higher titer. As the HMW can be easily removed in the downstream process, the slight increase in the HMW proportion does not affect a high yield of purified bsAb in the knockout clone. Therefore, hindering the H3K9me3 deposition by eliminating the ATF7IP-SETDB1 complex led transcriptional de-repression of transgene, resulting in improvement of productivity of CHO-bsAb cells.

### EXAMPLE 4. INCREASE IN PRODUCTIVITY OF MAB PRODUCING CHO CELL BY KNOCKOUT OF ATF7IP AND SETDB1

To identify whether knockout of Atf7ip and Setdb1 impacts CHO cells producing relatively easy-to-express proteins, the effect of the knockout of Atf7ip and Setdb1 in the CHO cell line producing rituximab (CHO-mAb) was examined.

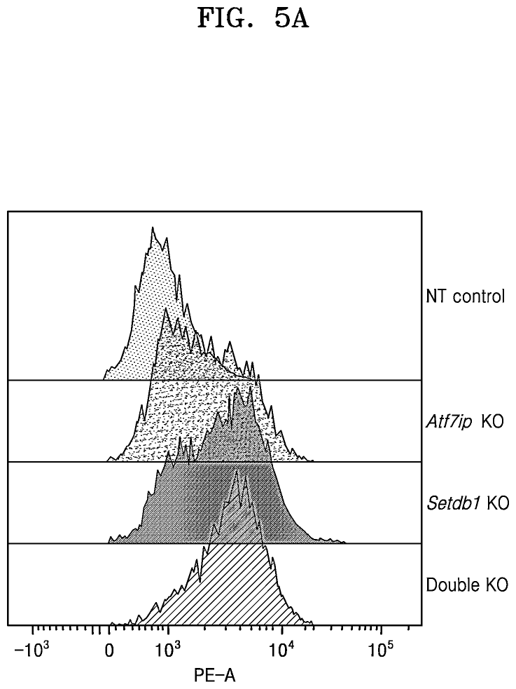

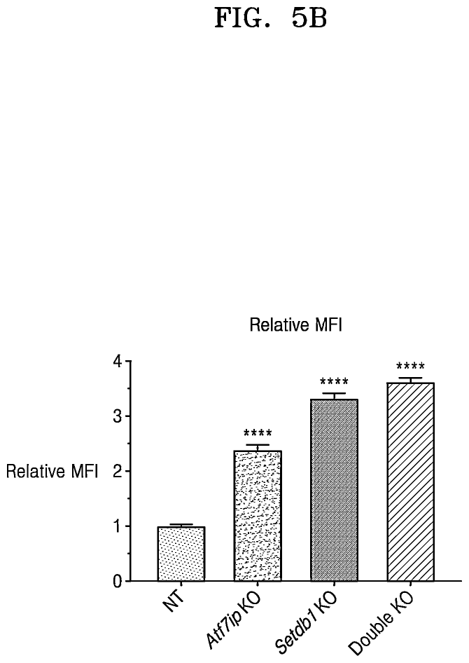

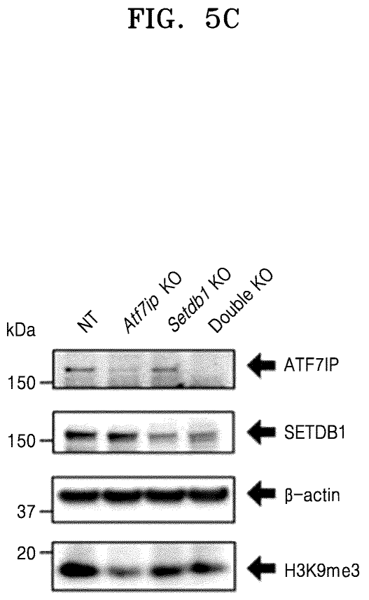

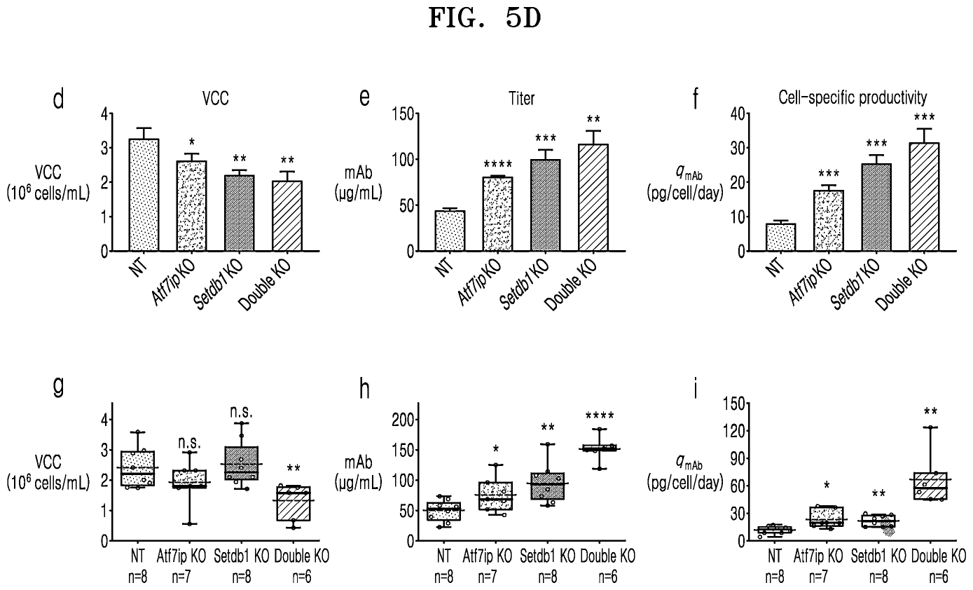

The Atf7ip knockout, Setdb1 knockout, and Atf7ip/Setdb1 double knockout cell pools were prepared in the same method as that employed for the CHO-bsAb cells in Example 3. All three knockout cell pools showed significantly higher fluorescence intensity than the NT control, and surprisingly, the extent of increase was much greater than that observed in the CHO-bsAb cells (FIG. 5A). The mean fluorescence intensity of Atf7ip knockout, Setdb1 knockout, and Atf7ip/Setdb1 double knockout cell pools was 2.4 times, 3.3 times, and 3.6 times higher than that of the NT control, respectively (FIG. 5B). Knockout of each target gene was validated by TIDE analysis and western blot analysis (FIG. 5C). Consistent with the results from the CHO-bsAb cells all three knockout cell pools showed a decrease in H3K9me3 level by varying degrees as compared to that of the NT control. However, unlike the CHO-bsAb cells, the SETDB1 level was not decreased in the CHO-mAb cells even after the ATF7IP was knocked out, and vice versa.

### 4.1. Cell Growth and mAb Titer

To evaluate the effect of gene knockout on cell growth and mAb production, the knockout cell pools were cultured in a 6-well plate and VCC and mAb titers were measured on day 3. In comparison with the NT control, all three knockout cell pools showed a decrease in VCC (d in FIG. 5D) and a significant increase in titer (e in FIG. 5D). The cell-specific mAb productivity (q_{mAb}) of Atf7ip knockout (17.9±1.3 pg/cell/day), Setdb1 knockout (25.4±2.4 pg/cell/day), and Atf7ip/Setdb1 double knockout (31.8±3.9 pg/cell/day) cell pools was higher than that of the NT control (8.1±0.7 pg/cell/day) by 2.2 times, 3.1 times, and 3.9 times, respectively (f in FIG. 5D). By limiting dilution, isogenic clones of the Atf7ip knockout, Setdb1 knockout, and Atf7ip/Setdb1 double knockout were generated. Indels of each clone were evaluated by TIDE analysis. All clones with verified gene disruption were cultured in a 6-well plate, and then VCC and mAb titer were measured on day 3. While Atf7ip/Setdb1 double knockout clone showed a significant decrease in VCC, VCC for Atf7ip knockout clone and Setdb1 knockout clone was not substantially different from that of the NT control clone (g in FIG. 5D). However, the mean titers and q_{mAb} were significantly increased in all three knockout clones, which is consistent with the results observed in the knockout cell pools (h and I in FIG. 5). In conclusion, elimination of the ATF7IP-SETDB1 complex also increased q_{mAb} in CHO-mAb cells.

### 4.2. Productivity and mRNA Expression Level

To further characterize the effect of Atf7ip and Setdb1 knockout on production of mAb in a batch culture, the highest producing clones were cultivated in a 125 mL Erlenmeyer flask containing 30 mL of culture medium as described in Example 3.2.

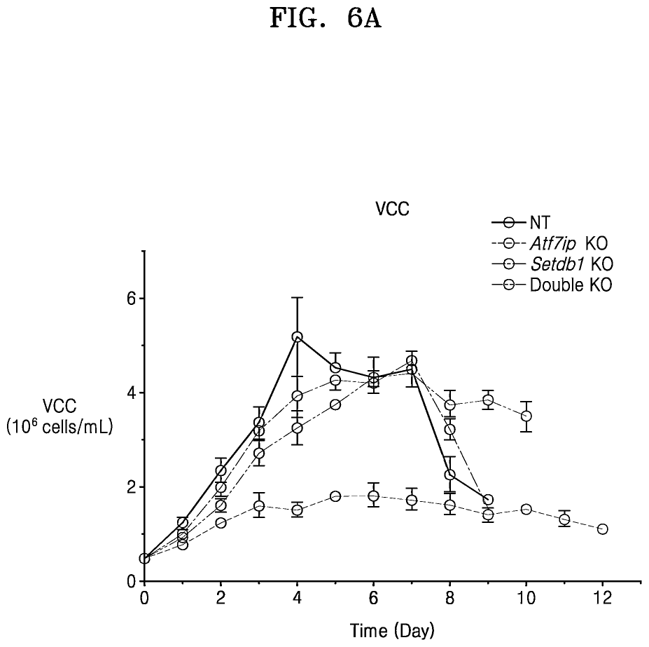

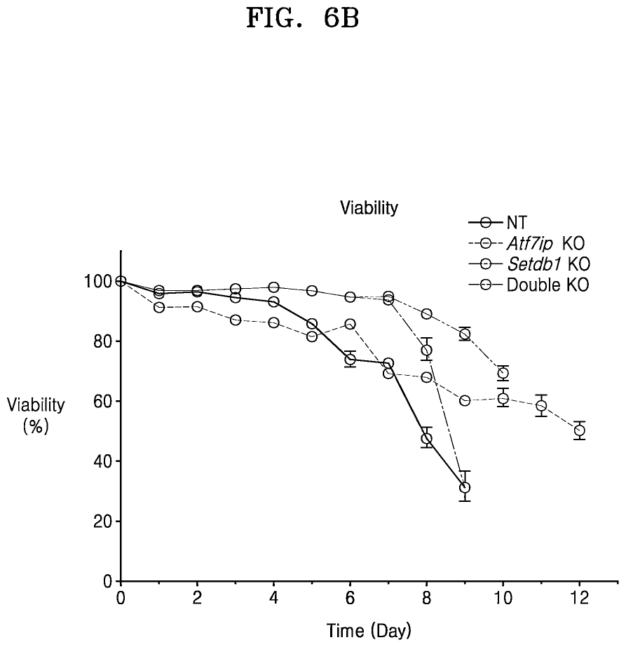

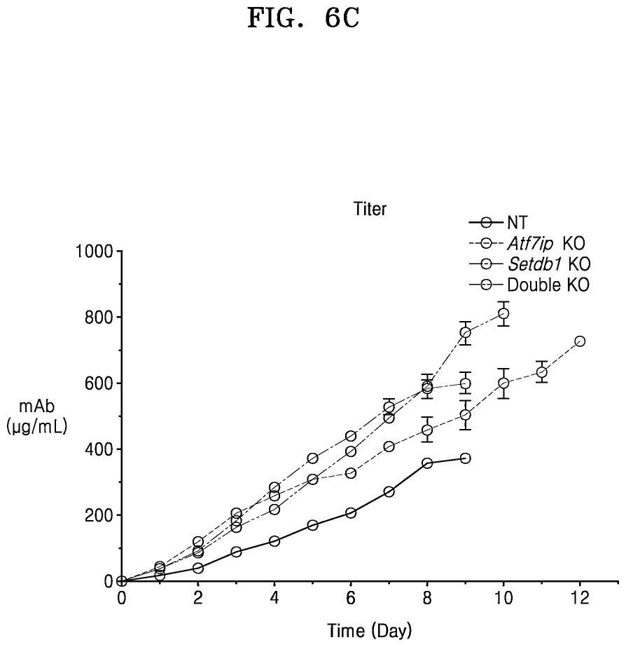

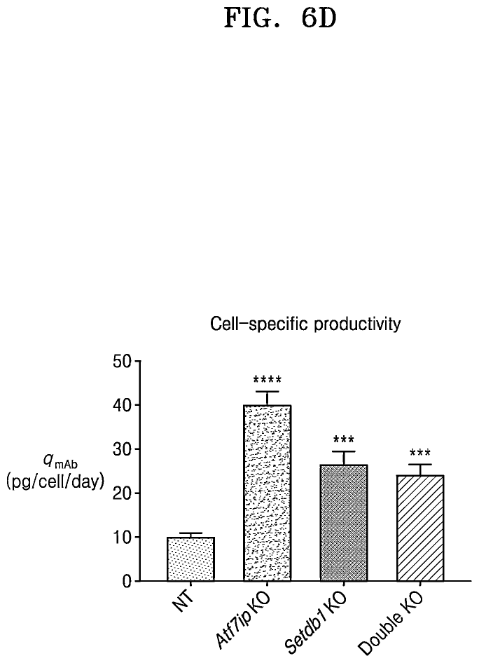

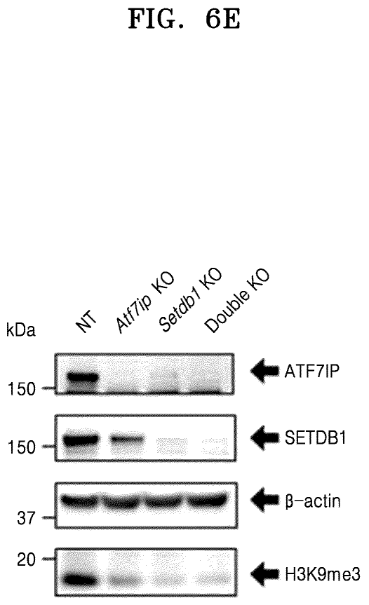

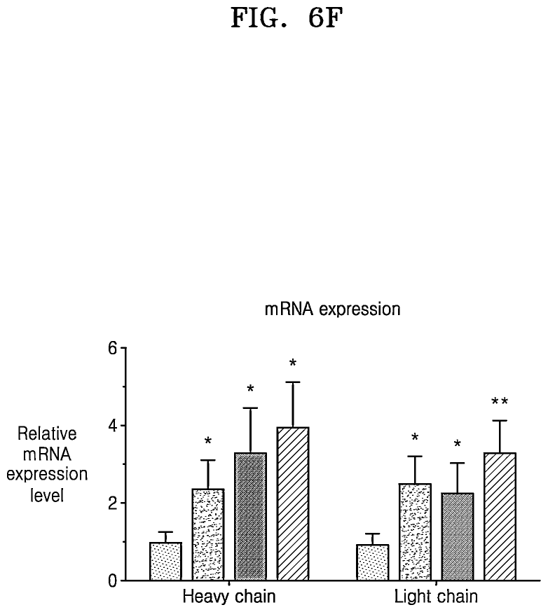

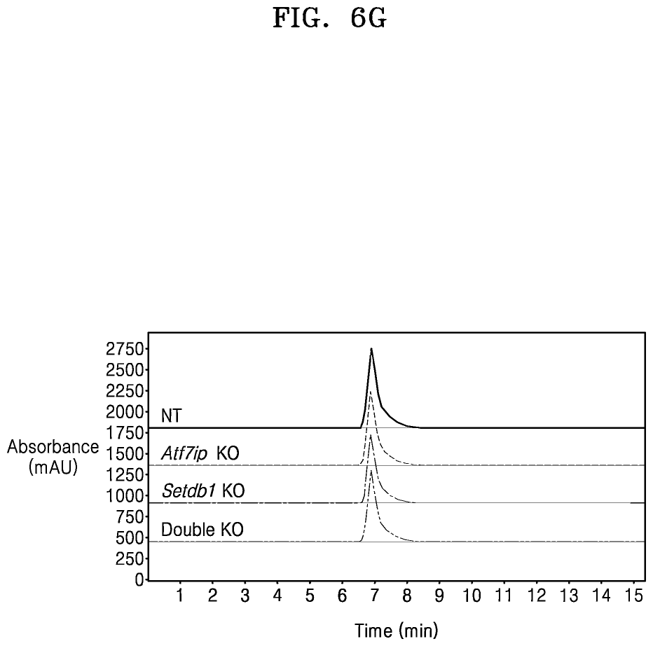

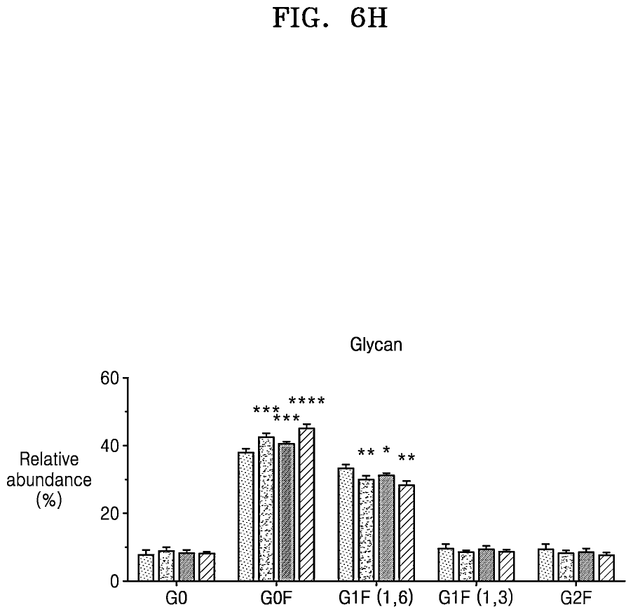

In comparison with the NT control, cell growth decreased by different degrees in the knockout clones, but cell viability was maintained longer for Atf7ip knockout and Atf7ip/Setdb1 double knockout clone (FIGS. 6A and 6B). In line with previous results, the knockout clones exhibited higher mAb production (FIG. 6C). The maximum mAb concentrations in the Atf7ip knockout (727.7±11.4 μg/mL), Setdb1 knockout (600.8±33.3 μg/mL), and Atf7ip/Setdb1 double knockout (810.5±36.3 μg/mL) clones were higher than that of the NT control (372.5±17.2 μg/mL) by 2.0 times, 1.6 times, and 2.2 times, respectively. The q_{bsAb} of the Atf7ip knockout (40.3±2.9 pg/cell/day), Setdb1 knockout (26.7±2.7 pg/cell/day), and Atf7ip/Setdb1 double knockout (24.5±2.0 pg/cell/day) clones from day 2 to day 5 were higher than that of the NT control (10.2±0.7 pg/cell/day) by 3.9 times, 2.6 times, and 2.4 times, respectively (FIG. 6D).

In addition, as described in Example 3.3, western blot analysis showed that each gene in each clone was successfully knocked out (FIG. 6E). The H3K9me3 level was reduced in all of the knockout clones. Relative mRNA expression levels of heavy chain and light chain were measured. The knockout clones showed heavy chain mRNA expression levels higher than those of the NT control by 2.4 to 3.9 times and light chain mRNA expression levels higher than those of the NT control by 2.4 to 3.4 times (FIG. 6F).

### 4.3. Quality of Antibody Produced by Knockout Cell

To identify the effect of the knockout of the ATF7IP-SETDB1 complex on quality of products, aggregation and N-glycosylation of Rituximab, the product of the CHO-mAb cells, were assessed.

### SEC

The mAb, Rituximab produced by the knockout cells were purified by protein A chromatography using a HiTrap Fibro™ PrismA (Cytiva) and a chromatography system (AKTA pure; Cytiva). The purified Rituximab was subjected to SEC by using an Agilent Technologies AdvanceBio SEC column (300 Å, 2.7 μm, 4.6×300 mm, Agilent Technologies) and analyzed by using an UHPLC system A (1290 Infinity II Bio LC, Agilent Technologies). A mobile phase consisted of 150 mM sodium phosphate, pH of 7.0 buffer. Signals were monitored at a wavelength of 220 nm.

Aggregation was not detected, showing a single peak of Rituximab in SEC (FIG. 6G).

### Analysis of N-Linked Glycans of Rituximab

The N-linked glycans of the purified Rituximab were analyzed by using a hydrophilic interaction HPLC column. Briefly, the N-linked glycans were released with PNGase F treatment (Roche Diagnostics, Basel, Switzerland) and labeled with 2-aminobenzamide (2-AB; Sigma-Aldrich). The resulting mixture was separated by using an AdvanceBio Glycan Map column (1.8 μm, 2.1×150 mm, Agilent Technologies) and analyzed by using a UHPLC system (1290 Infinity II Bio LC, Agilent Technologies). The 2-AB-derived N-glycans were monitored by using a fluorescence detector with excitation and emission wavelengths of 260 nm and 430 nm, respectively.

While there was no change in G0 and G2F glycan, slight changes were observed in G0F and G1F glycans (FIG. 6H).

Collectively, the data suggest that targeting the ATF7IP-SETDB1 complex could be a promising strategy for cell engineering to enhance productivity in therapeutic protein-producing CHO cells.

The recombinant animal cell in which expression of at least one of the Atf7ip gene and the Setdb1 gene is suppressed or eliminated according to the present disclosure shows increase in expression of a gene encoding a product of interest such as a therapeutic protein, thereby significantly enhancing productivity of the product of interest and may be efficiently used for production of biopharmaceuticals such as gene therapy products, cell therapy products, and vaccines, as well as the therapeutic proteins.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A recombinant animal cell having knockout of at least one of Atf7ip gene and Setdb1 gene.

2. The recombinant animal cell of claim 1, wherein both the Atf7ip gene and the Setdb1 gene are knocked out.

3. The recombinant animal cell of claim 1, wherein the recombinant animal cell comprises a gene encoding a product of interest.

4. The recombinant animal cell of claim 3, wherein the recombinant animal cell exhibits increased productivity of the product of interest as compared to a parent cell from which the recombinant animal cell is derived with at least one of the Atf7ip gene and the Setdb1 gene knocked out.

5. The recombinant animal cell of claim 3, wherein the product of interest is a therapeutic protein, a gene therapy product, a cell therapy product, a viral vector, or a viral antigen.

6. The recombinant animal cell of claim 5, wherein the product of interest is an antibody.

7. The recombinant animal cell of claim 6, wherein the antibody is a bispecific antibody.

8. The recombinant animal cell of claim 3, wherein the gene encoding a product of interest is inserted into a genome of the recombinant animal cell or is present as a vector.

9. The recombinant animal cell of claim 3, wherein the product of interest is produced in the cell and secreted from the cell.

10. The recombinant animal cell of claim 1, wherein the recombinant animal cell is a Chinese hamster ovary (CHO) cell.

11. A method of producing a product of interest, the method comprising:
culturing a recombinant animal cell having a gene encoding a product of interest, and knockout of at least one of an Atf7ip gene and a Setdb1 gene; and
recovering the product of interest from a culture of the recombinant animal cell.

12. The method of claim 11, wherein the product of interest is a therapeutic protein, a gene therapy product, a cell therapy product, a vector, or a vaccine.

13. The method of claim 11, wherein the recombinant animal cell produces a larger amount of the product of interest than a parent cell from which the recombinant animal cell is derived with at least one of the Atf7ip gene and the Setdb1 gene knocked out.

14. The method of claim 11, wherein the recombinant animal cell is a Chinese hamster ovary (CHO) cell, and the product of interest is an antibody.

15. The method of claim 11, wherein the recombinant animal cell is a CHO cell, and the product of interest is produced in the cell and released from the cell.
